(19) United States
(12) Patent Application Publication
(10) Pub. No.: US 2003/0236509 A1
(43) Pub. Date: Dec. 25, 2003

(54) Colostomy bag with gas release valve and method for releasing gas collected in the colostomy bag

(76) Inventor: Edgar Ignacio Silvestrini, Chicago, IL (US)

Correspondence Address: PATENTS+TMS, A Professional Corporation, 1914 North Milwaukee Avenue, Chicago, IL, 60647, US

(21) Appl. No.: 10/180,615

(22) Filed: Jun. 25, 2002

(51) Int. Cl. A61F 5/44

(52) U.S. Cl. 604/332

(57) ABSTRACT

A colostomy bag with a release valve and a method for releasing gas from the colostomy bag is provided. The release valve is attached to a colostomy bag for release of gas in the colostomy bag. The gas may be released manually by a person with the use of one hand. Accordingly, a person who is disabled or otherwise incapable of using two hands, may release gas from the colostomy bag and prolong the use of a single bag by extending the time it may take for the bag to fill. In addition, a method for releasing gas collected in a colostomy bag is provided such that the frequency of replacing and/or intermittently cleaning the colostomy bag is reduced.

### BACKGROUND OF THE INVENTION

The present invention generally relates to a colostomy bag with a release valve and a method for releasing gas collected in the colostomy bag. More specifically, the present invention relates to a gas release valve attached to a colostomy bag for releasing gas trapped in the bag. In addition, the present invention relates to a method for releasing gas collected in a colostomy bag such that replacement and/or cleaning of the colostomy bag is reduced.

After the surgical removal of the large intestine of a patient due to, for example, colon cancer, severe acute obstruction, trauma, gunshot wounds, and/or the like, a patient may require a means for removing bodily waste. A colostomy bag is generally used by a patient after the surgical removal of the large intestine performed during a colostomy. A colostomy is a surgical procedure in which an artificial permanent opening, referred to as a stoma, is provided and through which the colon may be artificially evacuated. Bodily waste exits the body by passing through the stoma into a colostomy bag. After the bag is full, the bag is usually detached from the stoma, discarded, and replaced. The frequent removal and replacement is burdensome, inconvenient, and often irritating to the patient.

Further, the colostomy bag is generally fitted with a clamp such that the waste may be removed while the bag is still attached to the body of the user. During use, gases and other waste from the body fill the bag and may be released through removal of the clamp. The gas causes the bag to blow up and becomes uncomfortable and very bulky when worn by the user. The gas can be so great as to pull the bag away from the body. This causes discomfort to the user and/or exposes the user to infection.

The discomfort caused by known colostomy bags limits the mobility of the user. The user may often become fearful of moving about because of a perceived embarrassment and discomfort should the bag be dislodged. Further, the known colostomy bags do not enable easy and/or convenient discharge of waste. For example, a person who is outdoors, or otherwise does not have access to a restroom or private area, may be inhibited from removing waste and/or cleaning the known colostomy bag. Because of the perceived embarrassment, discomfort, and other inconveniences of the known colostomy bags, the mobility of the user may become limited.

Further, a colostomy bag generally requires at least two hands for handling during cleaning and/or removal and changing of the bag. Accordingly, many patients that use the known colostomy bag require assistance. For example, persons that are bedridden, paralyzed due to, for example, a stroke or spinal cord injuries, have limited use of their upper extremities, are missing an arm, or the like need assistance handling the known colostomy bag.

A need, therefore, exists for a colostomy bag with a valve for release of gas and a method for releasing gas such that the colostomy bag may not need to be cleaned and/or frequently changed. Further, a need exists for a colostomy bag and method for releasing gas from the colostomy bag wherein the colostomy bag is easier to handle.

### SUMMARY OF THE INVENTION

The present invention generally relates to a colostomy bag with a release valve and a method for releasing gas collected in the colostomy bag More specifically, the present invention relates to a gas release valve attached to a colostomy bag for allowing release of gas that gets trapped in the bags. In addition, the present invention relates to a method for releasing gas collected in a colostomy bag such that replacement and/or cleaning of the colostomy bag is reduced.

To this end, in an embodiment of the present invention, a colostomy bag for connection to a stoma of a user wherein the user has human waste discharged through the stoma is provided. The colostomy bag has a container having walls defining an interior and an opening in one of the walls of the container. The opening attaches to the stoma. In addition, the colostomy bag has a release valve in communication with the interior of the container.

In an embodiment, an open position associated with the release valve permits gas to pass from the interior of the container to an area outside of the container.

In an embodiment, a closed position associated with the release valve prevents gas from the interior to escape to an area outside of the container.

In an embodiment, the opening may be increased to fit the stoma.

In another embodiment of the present invention, a method for releasing gas collected in a colostomy bag of a patient is provided. The method comprises the steps of: providing a container having walls defining an interior, a release valve and a first opening in the walls of the container; attaching the first opening to a stoma of the patient; receiving waste from the patient into the interior of the container; and releasing gas from the interior of the container.

In an embodiment, the method comprises an additional step of increasing a size of the first opening to receive the stoma of the patient.

In an embodiment, the method comprises an additional step of applying pressure to the release valve.

In an embodiment, the method comprises an additional step of removing a cap from the release valve.

In an embodiment, the method comprises an additional step of replacing the container.

In another embodiment of the present invention, a colostomy bag for connection to a stoma of a user wherein the user has human waste discharged through the stoma is is provided. The colostomy bag has a container having walls defining an interior and a release valve in communication with the interior of the container. In addition, the colostomy bag has a first opening and a second opening. The first opening is in one of the walls of the container and the first opening attaches to the stoma. The second opening is in one of the walls of the container and the second opening drains the container.

In an embodiment, an open position associated with the release valve permits gas to pass from the interior of the container to an area outside of the container.

In an embodiment, a closed position associated with the release valve prevents gas from the interior to escape to an area outside of the container.

In an embodiment, a seal is associated with the second opening of the container.

In an embodiment, the second opening of the container may be opened and closed at will.

In an embodiment, the first opening may be sized to fit the stoma.

It is, therefore, an advantage of the present invention to provide a colostomy bag with a release valve and a method for releasing gas collected in the colostomy bag that is easy and/or convenient to use.

Another advantage of the present invention is to provide a colostomy bag with a release valve and a method for releasing gas collected in the colostomy bag wherein a person using the bag may release gas from the valve with one hand.

Another advantage of the present invention is to provide a colostomy bag with a release valve and a method for releasing gas collected in the colostomy bag wherein gas may be released from the colostomy bag by an individual during any activity of the individual, such as, for example, walking, running, driving, riding a bicycle, motorcycle, in a boat, in an airplane, and the like.

Further, an advantage of the present invention is to provide a colostomy bag with a release valve and a method for releasing gas collected in the colostomy bag wherein the workload of nurses in hospitals or other healthcare professionals is reduced.

Still further, an advantage of the present invention is to provide a colostomy bag with a release valve and a method for releasing gas collected in the colostomy bag wherein the need to replace and/or intermittently clean the colostomy bag is reduced.

Another advantage of the present invention is to provide a colostomy bag with a release valve and a method for releasing gas collected in the colostomy bag wherein fewer bags are used by a patient and the cost of using the bags is thereby reduced.

Yet another advantage of the present invention is to provide a colostomy bag with a release valve and a method for releasing gas collected in the colostomy bag wherein replacement of the colostomy bag is reduced.

Additional features and advantages of the present invention are described in, and will be apparent from, the detailed description of the presently preferred embodiments and from the drawings.

### BRIEF DESCRIPTION OF THE DRAWINGS

FIG. 1 illustrates a perspective view of a colostomy bag having a gas release valve in an embodiment of the present invention.

FIG. 2A illustrates a cross-sectional view of a gas release valve in a closed position in an embodiment of the present invention.

FIG. 2B illustrates a cross-sectional view of a gas release valve in an open position in an embodiment of the present invention.

FIG. 2C illustrates a perspective view of a gas release valve in an embodiment of the present invention.

FIG. 3 illustrates a flowchart for a method for releasing gas collected in a colostomy bag in an embodiment of the present invention.

### DETAILED DESCRIPTION OF THE PRESENTLY PREFERRED EMBODIMENTS

The present invention generally relates to a colostomy bag with a release valve and a method for releasing gas collected in the colostomy bag. More specifically, the present invention relates to a gas release valve attached to a colostomy bag for release of gas in the bag. In addition, the present invention relates to a method for releasing gas collected in a colostomy bag such that replacement and/or cleaning of the colostomy bag is reduced.

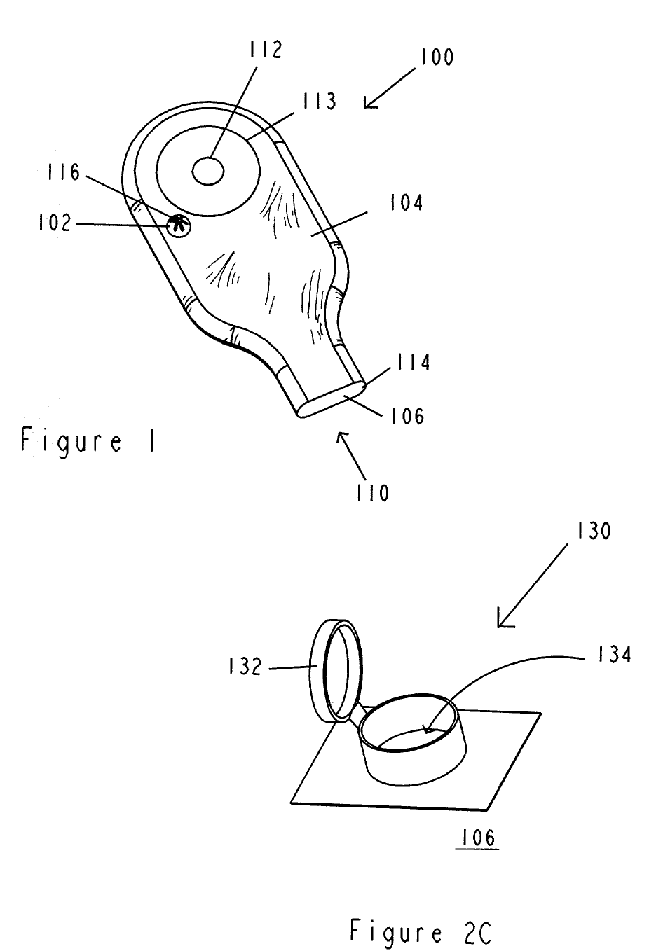

Referring now to the drawings wherein like numerals refer to like parts, FIG. 1 generally illustrates a colostomy bag 100 having a gas release valve 102. The colostomy bag 100 may have an exterior surface 104 and an interior 106. The colostomy bag 100 may preferably be constructed with a reuseable, liquid impermeable material, such as, for example, plastic or the like. The colostomy bag 100 is generally illustrated in FIG. 1 having an oblong shape. However, the colostomy bag 100, may be constructed in the shape of a sphere, square, or the like. Further, the colostomy bag 100 may be constructed from any one or more of various materials apparent to those skilled in the art without departing from the spirit of the present invention.

The colostomy bag 100 may have an opening 112 on the exterior surface 104. The opening 112 may be configured for receiving human waste via a stoma. A stoma is an artificial permanent opening in the abdominal wall created, for example, during a surgical procedure. The opening 112 may be increased in diameter to adjust the opening 112 for the stoma of a particular patient. The opening 112 may be increased in diameter by simply cutting along the edge of the opening 112 with scissors, a knife, or the like (not shown). For example, the opening 112 may be increased in size to an extent indicated by a diameter 113 illustrated in FIG. 1.

The colostomy bag 100 may receive human waste from, for example, a large intestine of the patient. The human waste may be evacuated from the patient, through the stoma and through the opening 112 to the interior 106 of the colostomy bag 100. The waste may continue to be collected in the colostomy bag 100 until the colostomy bag 100 is full. After the colostomy bag 100 is full, the colostomy bag 100 may be removed and/or replaced. Of course, the colostomy bag 100 may be removed and/or replaced prior to the colostomy bag 100 being filled.

The colostomy bag 100 may further have a bottom end 110 located a distance from the opening 112. The bottom end 110 of the colostomy bag 100 may have a second opening 114 through which contents received in the interior 106 of the colostomy bag 100 may be emptied. For example, after the colostomy bag 100 is full, the colostomy bag 100 may be removed and the contents therein may be emptied through the second opening 114 of the bottom end 110 of the colostomy bag 100. The second opening 114 may be releasably closed with a clip, adhesive, zipper, or other device known in the art.

Alternatively, the second opening 114 may be permanently sealed. Further, the colostomy bag 100 may be constructed with only one opening, such as, for example, the opening 112, i.e. without the second opening 114. Of course, if the second opening 114 is permanently sealed, or if the colostomy bag 100 is constructed with only one opening, the contents received in the interior 106 of the colostomy bag 100 may not be emptied. The colostomy bag 100 having only one opening may not be emptied and reused but simply discarded and replaced.

The release valve 102 may be a one-way valve such that gas may be released from the interior 106 of the colostomy bag 100. The release valve 102 of the present invention may permit movement of gas in one direction, namely from the interior 106 of the colostomy bag 100 to an area outside of the colostomy bag 100. The release valve 102 may be defined by any mechanical device by which the flow of gas may be started, stopped, or regulated by opening, shutting, or partially obstructing a passageway from the colostomy bag 100 to an area outside of the colostomy bag 100.

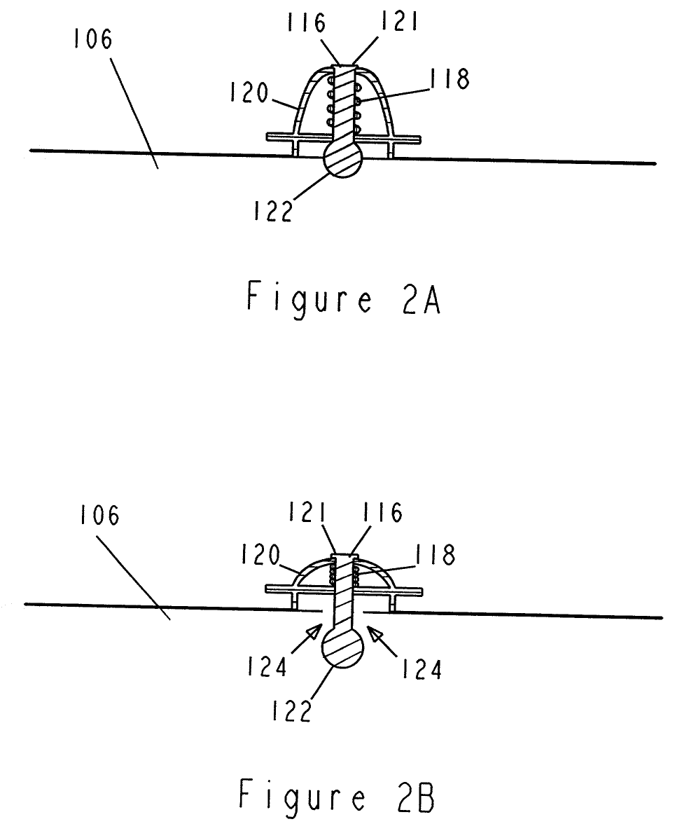

For example, in an embodiment of the present invention, an example of a suitable release valve 102 of the colostomy bag 100 is generally illustrated in FIGS. 2A and 2B. The release valve 102 generally illustrated in FIG. 2B is in an open position. The release valve 102 may have a release button 116 and a spring 118 housed within a flexible cover 120. Preferably, the cover 120 is constructed from a flexible material. The release button 116 may have a flat top 121 having a diameter “C”. The release button 116 may further have a sphere-shaped bottom 122 having a diameter “D”. The diameter “D” may be greater than the diameter “C” of the flat top 120. A user may apply pressure to the release button 116 to compress the spring 118. By compressing the spring 118, the sphereshaped bottom 122 of the release button 116 may be forced to further protrude into the interior 106 of the colostomy bag 100. After the sphere-shaped bottom 122 of the release button 116 is forced to further protrude into the interior 106 of the colostomy bag 100, a gap 124 may be created. The gap 124 may allow the gas (not shown) to escape the interior 106 of the colostomy bag 100.

Referring to FIG. 2A, the release valve 102 is generally illustrated in a closed position. After the user releases pressure on the release button 116, the spring 118 may expand. After the spring 118 expands, the sphereshaped bottom 122 of the release button 116 may close the gap 124. Accordingly, the sphere-shaped bottom 122 of the release button 116 may seal the interior 106 of the colostomy bag 100 as shown in FIG. 2A.

Referring to FIG. 2C, in another embodiment of the present invention, a release valve 130 may have a cap 132. The cap 132 may be removed to expose an opening 134 to the interior 106 of the bag wherein gas may be released to the atmosphere. Further, one of any number of different release valves apparent to those skilled in the art may be incorporated with the colostomy bag 100 without departing from the spirit of the present invention.

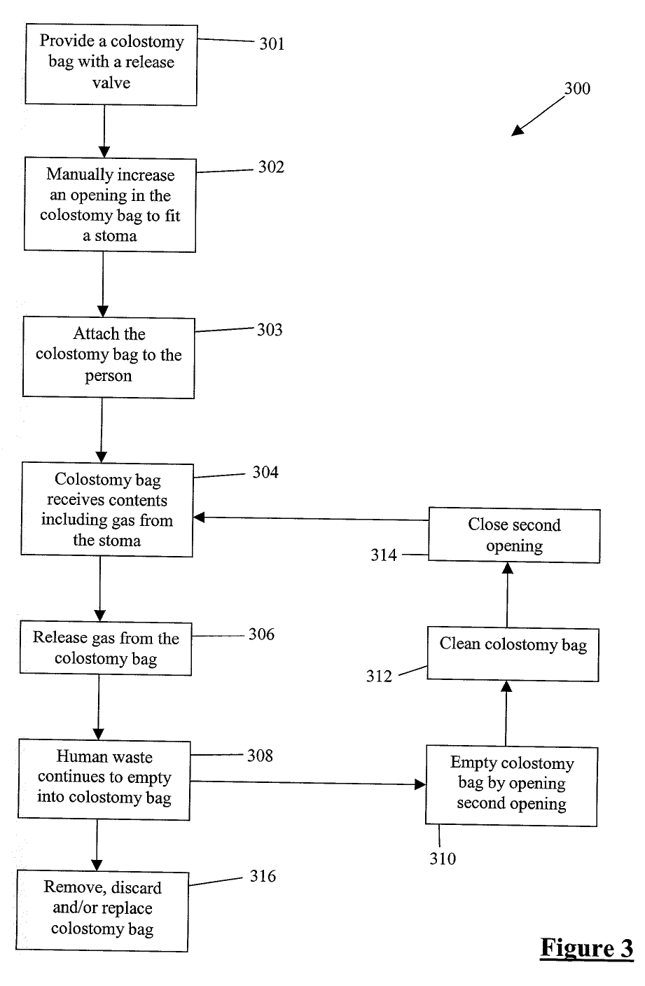

Referring now to FIG. 3, a flowchart 300 for a method for releasing gas collected in a colostomy bag such that the frequency of replacing and/or cleaning the colostomy bag 100 is reduced is generally illustrated. First, the colostomy bag 100 having the release valve 102, the top end 108, and the opening 112 is provided as shown at step 301. A diameter of the opening 112 of the colostomy bag 100 may be manually increased as needed to receive the stoma as shown at step 302. After the colostomy bag 100 is attached to a person as shown at step 303, the colostomy bag 100 may receive contents from the stoma as shown at step 304.

The contents received by the colostomy bag 100 from the stoma are human waste including gas. The human waste is received from the large intestine of the person. The person may not be able to control the amount of waste or when that waste is caused to exist. Accordingly, a person may be, for example, in a public area, a train, an airplane, a park, or the like, when the colostomy bag 100 involuntarily receives the human waste, including gas. While on an airplane, in a public area, or the like, changing, cleaning, or otherwise emptying the colostomy bag 100 may be difficult and cumbersome.

However, after the colostomy bag 100 receives contents from the stoma, including gas, the gas may be released from the colostomy bag 100 as shown at step 306. The person may release the gas by applying pressure to the release button 116 of the release valve 102. Alternatively, a person may release the gas from the colostomy bag 100 by removing a cap 132 of the release valve 130. Of course, the gas may be released by use of any known release valve incorporated with the colostomy bag 100. By releasing the gas, the colostomy bag 106 may not become full as quickly, providing the person with additional time before having to clean, replace, or otherwise empty the colostomy bag 100. Human waste may continue to involuntarily fill the colostomy bag 100 as shown at step 308.

After the colostomy bag is full, the human waste received in the colostomy bag 100 may be emptied by releasing the sealed second opening 114 as shown at step 310. The colostomy bag 100 may be cleaned as shown at step 312, and the second opening 114 may be closed again as shown at step 314. Alternatively, after the colostomy bag 100 is full, the colostomy bag 100 may be removed, discarded, and/or replaced as shown at step 316.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

I claim:

1. A colostomy bag for connection to a stoma of a user wherein the user has human waste discharged through the stoma, the bag comprising:
a container having walls defining an interior;
an opening in one of the walls of the container wherein the opening attaches to the stoma; and
a release valve in communication with the interior of the container.

2. The colostomy bag of claim 1 further comprising:
an open position associated with the release valve wherein the open position permits gas to pass from the interior of the container to an area outside of the container.

3. The colostomy bag of claim 1 further comprising:
a closed position associated with the release valve wherein the closed position prevents gas from the interior to escape to an area outside of the container.

4. The colostomy bag of claim 1 wherein the opening may be increased to fit the stoma.

5. A method for releasing gas collected in a colostomy bag of a patient, the method comprising the steps of:
providing a container having walls defining an interior, a release valve and a first opening in the walls of the container;
attaching the first opening to a stoma of the patient;
receiving waste from the patient into the interior of the container; and
releasing gas from the interior of the container.

6. The method of claim 5 further comprising the step of:
increasing a size of the first opening to receive the stoma of the patient.

7. The method of claim 5 further comprising the step of:
applying pressure to the release valve.

8. The method of claim 5 further comprising the step of:
removing a cap from the release valve.

9. The method of claim 5 further comprising the step of:
replacing the container.

10. A colostomy bag for connection to a stoma of a user wherein the user has human waste discharged through the stoma, the bag comprising:
a container having walls defining an interior;
a first opening in one of the walls of the container wherein the first opening attaches to the stoma;
a second opening in one of the walls of the container wherein the second opening drains the container; and
a release valve in communication with the interior of the container.

11. The colostomy bag of claim 10 further comprising:
an open position associated with the release valve wherein the open position permits gas to pass from the interior of the container to an area outside of the container.

12. The colostomy bag of claim 10 further comprising:
a closed position associated with the release valve wherein the closed position prevents gas from the interior to escape to an area outside of the container.

13. The colostomy bag of claim 10 further comprising:
a seal associated with the second opening of the container.

14. The colostomy bag of claim 10 wherein the second opening of the container may be opened and closed at will.

15. The colostomy bag of claim 10 wherein the first opening may be sized to fit the stoma.
